(19) United States
(12) Patent Application Publication
(10) Pub. No.: US 2008/0124707 A1
(43) Pub. Date: May 29, 2008

(54) Nucleic acid concatenation

(75) Inventors: Yijun Ruan, Singapore (SG); Patrick Ng, Singapore (SG); Melissa Jane Fullwood, Singapore (SG); Yen Ling Lee, Singapore (SG)

Correspondence Address: ROTHWELL, FIGG, ERNST & MANBECK, P.C., 1425 K STREET, N.W., SUITE 800, WASHINGTON, DC, 20005, US

(73) Assignee: Agency for Science, Technology and Research, Singapore (SG)

(21) Appl. No.: 11/449,872

(22) Filed: Jun. 9, 2006

(51) Int. Cl. C12Q 1/68; C12P 19/34

(52) U.S. Cl. 435/6; 435/91.2

(57) ABSTRACT

The present invention provides a method of manipulating nucleic acids. In particular, of length-controlled concatenating of nucleotide fragments, the method comprising: (a) providing at least two nucleotide fragments, wherein each fragment has one ligatable end and one non-ligatable end; and (b) allowing the two fragments to ligate at the ligatable ends to form an oligonucleotide comprising at least two concatenated nucleotide fragments. The present invention also provides an isolated oligonucleotide comprising at least two nucleotide fragments, wherein each fragment has at least one ligatable end and and one non-ligatable end, and the fragments are ligated at the ligatable ends to form the oligonucleotide.

### FIELD OF THE INVENTION

The present invention generally relates to the field of nucleic acids. Specifically, the present invention relates to concatenation of nucleic acids.

### BACKGROUND OF THE INVENTION

One of the most important goals of the human genome project is to provide complete lists of genes for the genomes of human and model organisms. Complete genome annotation of genes relies on comprehensive transcriptome analysis by experimental and computational approaches. Ab initio predictions of genes must be validated by experimental data. However; due to the complexity and immense volume of transcripts expressed in the various developmental stages of an organism's life cycle, complete sequencing analysis of all different transcriptomes still remains unrealistic.

In the past decade this problem has been overcome with a cDNA tagging method in which partial sequences that represent full transcripts are obtained. This strategy has been widely used for determining genes and characterizing transcriptomes.

In the expressed sequence tag (EST) approach, cDNA clones are sequenced from 5′ and/or 3′ nucleotides (Adams, M., et al., 1991, Science, 252, 1651-1656). Each EST sequence read would generate; on average, a 500 bp tag per transcript. The number of identical or overlapping ESTs would indicate the relative level of gene expression activity. Though this is an effective approach to identifying genes, it is prohibitively expensive to tag every transcript in a transcriptome. In practice, sequencing usually ceases after 10,000 or fewer ESTs are obtained from a cDNA library where millions of transcripts might be cloned.

To increase the efficiency of sequencing and counting large numbers of transcripts, Serial Analysis of Gene Expression (SAGE) (Velculescu, V. E., et al., 1995, Science, 270, 484-487) and the recent Massively Parallel Signature Sequencing (MPSS) technique (Brenner S, et al., 2000, Nature Biotechnology, 18, 630-634) were developed based on how a short signature sequence (14-20 bp) of a transcript can be sufficiently specific to represent that gene.

Experimentally, one short tag per transcript can be extracted from cDNA. Such short tags can be sequenced efficiently either by a concatenation tactic (as for SAGE), or by a hybridization-based methodology for MPSS. For example; in SAGE, multiple tags are concatenated into long DNA fragments and cloned for sequencing. Each SAGE sequence readout can usually reveal 20-30 SAGE tags. A modest SAGE sequencing effort of less than 10,000 reads will have significant coverage of a transcriptome. Transcript abundance is measured by simply counting the numerical frequency of the SAGE tags.

In theory, short DNA tags of about 20 bp can be specifically mapped to a single location within a complex mammalian genome and uniquely represent a transcript in the content of whole transcriptome. However, in reality, there still exist a large number of “ambiguous” SAGE tags (14-21 bp) and MPSS tags (17 bp) that have multiple locations in a genome, and may be shared by many genes.

A recent sequencing technology is that of “pyrosequencing” or “454” technology (Margulies et al, 2005). In recently published findings, a “454” sequencing run can simultaneously read 300,000 templates and achieve a 100-fold efficiency increase and 10-fold cost reduction compared with current sequencing instruments. However; each “454” sequencing read can only read about 100 bp, seriously limiting its potential because it is difficult to sequence large contiguous stretches of DNA.

Hence; the main problems in the current art of genomic sequencing are of ambiguity when using short nucleotide tags to represent gene transcripts, and the limitation of current multiplex sequencing technologies to reading short stretches of nucleotides.

### SUMMARY OF THE INVENTION

The present invention solves the problems mentioned above by providing a new method of manipulating nucleic acids. More specifically, the present invention relates to manipulation of nucleic acids. In particular, the invention relates to methods for the preparation of nucleotide fragments by concatenation.

In one aspect, the present invention provides a new method of concatenation of nucleotide fragments. In particular, there is provided a length-controlled concatenation of nucleotide fragments such that concatemers having a desired number of nucleotide fragments; or having a particular length, may be prepared. The present invention also provides molecules and components prepared by the method. The nucleotide fragment according to the invention may comprise at least one ditag and/or at least one tag. Accordingly, a concatenation of at least two nucleotide fragments may comprise at least two concatenated ditags and/or tags

Accordingly, there is provided a method of length-controlled concatenating nucleotide fragments, the method comprising: (a) providing at least two nucleotide fragments, wherein each fragment has one ligatable end and one non-ligatable end; and (b) allowing the two fragments to ligate at the ligatable ends to form at least one oligonucleotide comprising of at least two concatenated nucleotide fragments.

In one embodiment, the method may further comprise the steps of treating the at least one oligonucleotide to produce at least one oligonucleotide having one ligatable end and one non-ligatable end, and allowing the oligonucleotide to ligate with a further oligonucleotide or a nucleotide fragment to form an oligonucleotide comprising more than two concatenated nucleotides. The method may be repeated one or more times to make at least one oligonucleotide with an increasing number of nucleotide fragments. According to one aspect, the repetition of concatenation yields a doubling of the number of concatenated nucleotide fragments.

The method may further comprise treating the at least one oligonucleotide to produce at least one oligonucleotide with two ligatable ends and allowing the oligonucleotide to self-circularize at the ligatable ends. The method may further comprise selecting the at least one circularized oligonucleotide and/or amplifying the oligonucleotide. The method may further comprise treating the circularized and/or amplified oligonucleotide to produce at least one oligonucleotide having one ligatable end and one non-ligatable end, and allowing at least two oligonucleotides to ligate at the ligatable ends to form a concatemer comprising at least two concatenated oligonucleotides.

In another embodiment, the method may further comprise: (a) treating the at least one oligonucleotide to produce at least one oligonucleotide having two ligatable ends compatible with each other, and allowing the oligonucleotide to self-circularize at its ligatable ends; (b) selecting the at least one self-circularized oligonucleotide; (c) optionally amplifying the selected oligonucleotide; (d) treating the oligonucleotide (from step b or c) to produce at least one oligonucleotide with one ligatable end and one non-ligatable end; and (e) allowing two oligonucleotides to ligate at the ligatable ends to form a concatemer comprising at least two concatenated oligonucleotides.

In another embodiment, the method comprises the steps of (a) providing at least one oligonucleotide, wherein the oligonucleotide has ligatable ends; (b) allowing the at least one oligonucleotide to self-circularize at its ligatable ends; (c) selecting the at least one self-circularized oligonucleotide; (d) treating the selected circularized oligonucleotide with at least one restriction enzyme to obtain at least one oligonucleotide with one ligatable end and one non-ligatable end; and (e) concatenating at least two oligonucleotides at the ligatable ends to form a concatemer of at least two oligonucleotides.

For the embodiments in this aspect of the invention, the nucleotide fragments, oligonucleotide(s) and/or concatemer(s) may be amplified. The amplification may be by bacterial amplification, by rolling circle amplification, and/or by polymerase chain reaction. The method may comprise repeating the steps one or more times to obtain concatemers having desired lengths and/or number of oligonucleotides or nucleotide fragments. The repeating may result in a doubling of the number of oligonucleotides in the concatemers. The ligatable end of each fragment may be a palindromic cohesive end. The ligatable end and/or the non-ligatable end may be located in at least one adaptor. The adaptor may be part of a plasmid or vector. The nucleotide fragment may comprise at least one ditag, the ditag comprising at least one first tag comprising a 5′ terminus and at least one second tag comprising a 3′ terminus of a polynucleotide. Each nucleotide fragment of the concatemer may have an orientation opposite to the orientation of a nucleotide fragment positioned upstream and/or downstream. The method may further comprise sequencing the concatemer. The sequencing may be by any suitable method, for example, by pyrosequencing.

In another aspect, the present invention provides an isolated concatemer (one oligonucleotide or at least two concatenated oligonucleotides) comprising at least two nucleotide fragments, wherein each fragment has at least one ligatable end and one non-ligatable end, and the fragments are ligated at the ligatable ends to form the concatemer. The ligatable ends may be palindromic cohesive ends. The fragment may comprise at least one ditag, the ditag comprising at least one first tag comprising a 5′ terminus and at least one second tag comprising a 3′ terminus of a polynucleotide. Each nucleotide fragment of the concatemer may have orientation opposite to the orientation of a nucleotide fragment positioned upstream and/or downstream. The concatemer may be inserted into a plasmid or vector. The polynucleotide may be DNA or RNA.

### BRIEF DESCRIPTION OF THE DRAWINGS

FIG. 1 illustrates the overview of one embodiment of the method of the present invention for preparing concatenated nucleotide fragments (concatements). Each nucleotide fragment comprises a ditag. The ditag comprises a first tag comprising a 5′ terminus (gray in the figure) and a second tag comprising a 3′ terminus (black in the figure) of a polynucleotide (for example, a full-length cDNA). The ditag may have one end sticky or ligatable and the other end non-sticky (FIG. 1A) or blunt-ended (may be either ligatable or non-ligatable) (FIG. 1B) The diPET as shown in FIG. 1A may be further ligated to nucleotide fragments with complementary ends and the diPET shown in FIG. 1B may be further ligated to nucleotide fragments with ligatable (blunt-ended, phosphorylated) ends. The two concatenated nucleotide fragments obtained comprise two concatenated ditags. In the figure, it is shown that the first and second ditag of each two concatenated nucleotide fragments have opposite orientation. The ditags are suitable for sequencing by large scale parallel sequencing methods, such as “454” sequencing. The ditags may be prepared by using single paired-end ditag (PET) plasmids or from insertion of other DNA sequences wherein the 5′ and 3′ termini are of interest. Mmel sites are present, but not shown on the plasmid.

FIG. 2 illustrates the overview of another embodiment of the method of the present invention for preparing concatemers comprising n-PETs (where n=4,8,16,32 . . . ), allowing the scaling up of the number of paired-end ditags (PETs). The oligonucleotides in the concatemers may be added in a precise manner as the capacity of the sequencing technology used increases. Two different types of adaptors are ligated to the ends of the diPET. These adaptors will contain the different restriction sites necessary for subsequent restriction digestion. The adaptors are ligated, such that only those diPETs with different adaptors ligated on will be circularized, and thus selected by an optional exonuclease treatment. Rolling circle amplification is performed to amplify the DNA. The DNA is then cut with the appropriate restriction enymes to generate a sticky end and an end that is not sticky, such that ligation may be used to form an n-PET. The cycle may then be repeated as desired to generate larger n-pets. Amplification and selection by PCR is also possible.

FIG. 3 illustrates electroeluted PETs in a PAGE gel. Lanes 1 and 9: Invitrogen 25 bp ladder. Lane 2: Invitrogen 100 bp ladder. Lane 3: BseR1 and BamH1 cut PETs from control library. Lane 4: BseR1 and BamH1 cut PETs from experimental library. Lane 5: 2 ul Invitrogen Low Mass ladder. Lane 6: 4 ul Invitrogen Low Mass ladder. Lane 7: Blunted BseR1 and BamH1 cut PETs from control library. Lane 8: Blunted BseR1 and BamH1 cut PETs from experimental library.

FIG. 4 illustrates electroeluted diPETs in a PAGE gel. Lane 1: Invitrogen 25 bp ladder. Lane 2: Invitrogen 100 bp ladder. Lane 3: Ligation product of control library; as expected, this library formed concatemers. Lane 4: Ligation product of experimental library—this library formed length-controlled diPETs, as can be seen by the single clear, sharp band. Lane 5: 2 ul of Invitrogen Low Mass ladder. Lane 6: 4 ul of Invitrogen Low Mass ladder.

FIG. 5 illustrates two examples of vectors used in the method of the present invention. FIG. 5A is the pGIS4a2 vector and FIG. 5B is the pGIS3h vector.

FIG. 6 illustrates concatenation of ditags to obtain a concatemer of a desired length for sequencing.

### SEQUENCE NUMBERING OF PRIMERS, ADAPTORS AND VECTORS

SEQ ID NO: 1: Gsul-oligo dT primer:
5′-GAGCTAGTTCTGGAGTTTTTTTTTTTTTTTTVN-3′
SEQ ID NO: 2: GIS-(N)6 adapter upper strand:
5′-CTAAACTCGAGGCGGCCGCGGATCCGACNNNNNN-3′
SEQ ID NO: 3: GIS-(N)6 adapter lower strand:
5′-p-GTCGGATCCGCGGCCGCCTCGAGTTT-3′
SEQ ID NO: 4: GIS-(N)5 adapter upper strand:
5′-CTAAACTCGAGGCGGCCGCGGATCCGACGNNNNN-3′
SEQ ID NO: 5: GIS-(N)5 adapter lower strand:
5′-p-GTCGGATCCGCGGCCGCCTCGAGTTT-3′
SEQ ID NO: 6: palindromic upper strand:
5′-GTCGGATCCGAC-3′
SEQ ID NO: 7: palindromic lower strand
5′-GTCGGATCCGAC-3′
SEQ ID NO: 8: n-PET TT-tailed adaptor (PMR 011)
upper strand:

| | |
|---|---|
| 5′ | (40 |
| GCTTGTAAGCTACTCCTCGATGTGCTGCAAGGCGATTAAG 3′ | nt) |

SEQ ID NO: 9: n-PET TT-tailed adaptor (PMR 011)
lower strand:

| | |
|---|---|
| 3′ | (42 |
| TTCGAACATTCGATGAGGAGCTACACGACGTTCCGCTAATTC | nt) |
| 5′ | |

SEQ ID NO: 10: n-PET TT-tailed adaptor (PMR 012)
upper strand:

| | |
|---|---|
| 5′ | (41 |
| GCTTGTAAGCTACTCCTCAGCGGATAACAATTTCACACAGG 3′ | nt) |

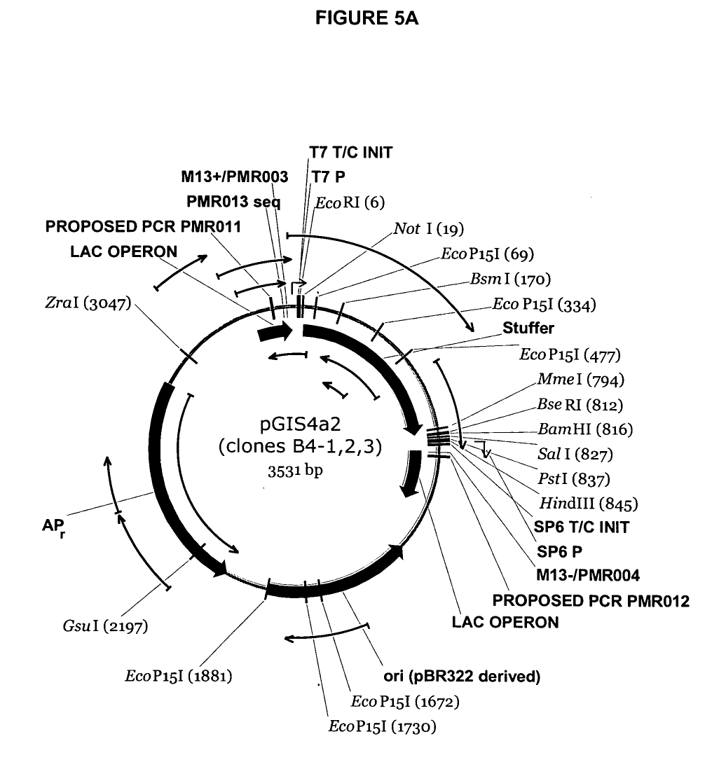

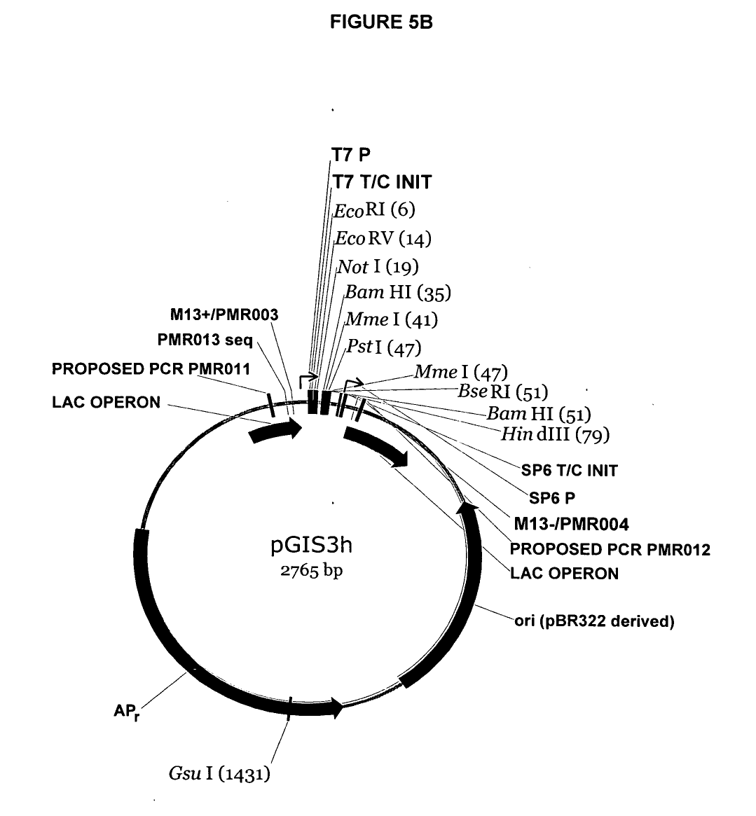

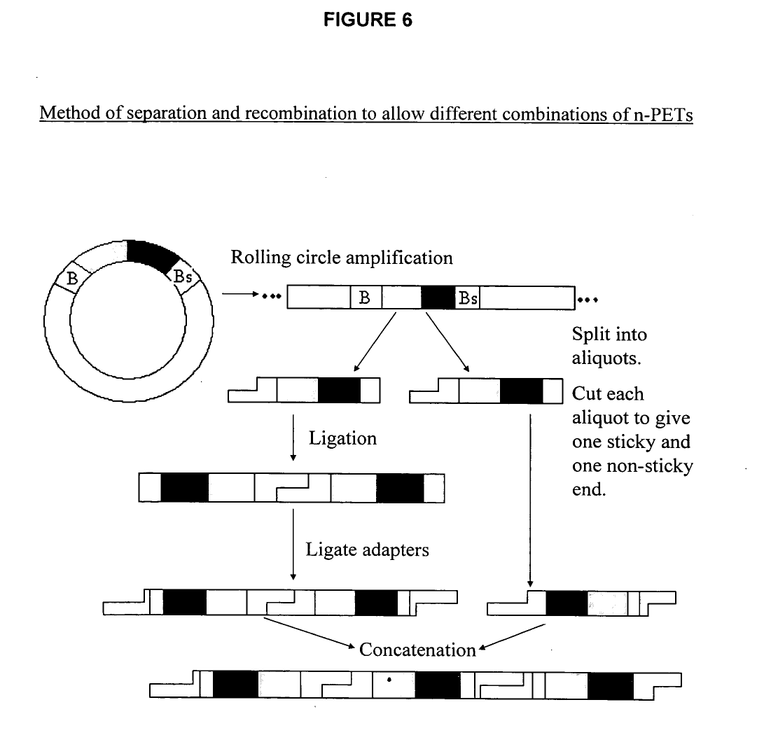

SEQ ID NO: 11: n-PET TT-tailed adaptor (PMR 012)
lower strand:

| | |
|---|---|
| 3′ | (43 |
| TTCGAACATTCGATGAGGAGTCGCCTATTGTTAAAGTGTGTCC | nt) |
| 5′ | |
| SEQ ID NO: 12: PMR011: | |
| 5′ GATGTGCTGCAAGGCGATTAAG 3′ | (22 |
| 5′ ends are phosphorylated | nt) |
| SEQ ID NO: 13: PMR012 | |
| 5′ AGCGGATAACAATTTCACACAGG 3′ | (23 |
| 5′ ends are phosphorylated | nt) |
| SEQ ID NO: 14: pGIS4a2 sense(FIG. 5A) | (3531 |
| See sequence listing. | bp) |
| SEQ ID NO: 15: pGIS4a2 antisense(FIG. 5A) | (3531 |
| See sequence listing. | bp) |
| SEQ ID NO: 16: pGIS3h sense (FIG. 5B) | (2765 |
| See sequence listing. | bp) |
| SEQ ID NO: 17: DGIS3h antisense (FIG. 5B) | (2765 |
| See sequence listing. | bp) |
| SEQ ID NO: 18; diPET from pGIS4a diPETting | (82 |
| sense | bp) |
| SEQ ID NO: 19; diPET from DGIS4a diPETting | (82 |
| antisense | bp) |
| SEQ ID NO: 20; diPET from DGIS3h diPETting | (90 |
| sense | bp) |
| SEQ ID NO: 21; diPET from DGIS3h diPETting | (90 |
| antisense | bp) |

### DETAILED DESCRIPTION OF THE INVENTION

### Definitions.

Restriction enzyme—A restriction enzyme (or restriction endonuclease) is an enzyme that cuts double-stranded DNA. The enzyme makes two incisions, one through each of the phosphate backbones of the double helix without damaging the bases. The chemical bonds cleaved by the enzymes may be reformed by other enzymes known as ligases, enabling restriction fragments obtained from different chromosomes or genes to be joined or spliced together, provided their ends are complementary or compatible. Type II enzymes recognize specific nucleic sequences (recognition sites) and cut DNA at defined positions close to or within their recognition sequence sites. They produce discrete restriction fragments and distinct gel banding patterns. Type IIs enzymes cleave outside of their recognition sequence to one side. MmeI, as well as most of the type IIs restriction enzymes, produce variable end lengths. Dunn et al (2002) showed that MmeI can cut 18/20 or 19/21 bases away in a rough proportion of 1:1. Type III enzymes are also large combination restriction-and-modification enzymes. They cleave outside of their recognition sequences and require two such sequences in opposite orientations within the same DNA molecule to accomplish cleavage. Homing endonucleases are rare double-stranded DNases that have large, asymmetric recognition sites (1240 base pairs) and coding sequences that are usually embedded in either introns (DNA) or inteins (proteins). Restriction enzymes may make cuts that leave either non-sticky (blunt) end or sticky (ligatable) ends with overhangs. A sticky-end fragment can be ligated not only to the fragment from which it was originally cleaved, but also to any other fragment with a complementary, compatible, cohesive or sticky end. As such, ends produced by different enzymes may also be compatible. Many type II restriction enzymes cut palindromic DNA sequences. If a restriction enzyme cuts a non-degenerate palindromic cleavage site, all the ends produced are compatible. A “palindromic” sequence is found where the sequence on one strand reads the same in the opposite direction on the complementary strand, allowing nucleic sequences cleaved to obtain palindromic cohesive ends can self-circularize when the two ends on the same strand mate. The meaning of “palindromic” in this context is different from its linguistic usage. For example, the sequence GTAATG is not a palindromic DNA sequence, while the sequence GTATAC is. Examples of restriction enzymes leaving cohesive or sticky ends include BamH1, EcoR1 and HindIII. An example of restriction enzymes leaving blunt, non-cohesive or non-sticky ends is AluI. Under the present invention, an end of a nucleic acid strand is said to be ligatable or capable of being ligated if it has a complementary, compatible, cohesive or sticky end or phosphorylated blunt end. An end of a nucleic is said not to be ligatable or not capable of being ligated if it and the other strand of nucleic acid both have dephosphorylated ends, or if it does not have an end that another strand of nucleic acid is complementary, compatible, cohesive or sticky to. Also, a restriction enzyme name (such as EcoR1) can also refer to the nucleic acid sequence or recognition site recognized by the enzyme as readily understood in the context in which the enzyme name or recognition site appears.

Nucleotide—a phosphoric ester of nucleoside; the basic structural unit of nucleic acids (DNA or RNA). Nucleotides form base pairs—one of the pairs of chemical bases joined by hydrogen bonds that connect the complementary strands of a DNA molecule or of an RNA molecule that has two strands; the base pairs are adenine with thymine and guanine with cytosine in DNA and adenine with uracil and guanine with cytosine in RNA. Nucleotides may be joined with or concatenated with other nucleotides. The term nucleotide may be used interchangeably with the term nucleic acid. Each nucleotide possesses a 5′ end and a 3′ end and accordingly, a strand of nucleic acids may also possess a 5′ end and a 3′ end. The end regions of a strand of nucleic acids may be referred to as the 5′ terminus and the 3′ terminus respectively. Nucleic acid sequences are conventionally read in the 5′ to 3′ direction which gives the orientation of the nucleotides. Short strands of nucleotides are referred to as oligonucleotides while longer strands are referred to as polynucleotides. Under the present invention, an oligonucleotide can comprise at least one nucleotide fragment, tag or ditag. A fragment is a length of nucleic acids obtained, derived or prepared from a longer length of nucleic acids. As such, a fragment can comprise at least one tag or ditag and can represent a larger nucleic acid molecule. Under the present invention, a polynucleotide can refer to a gene, a message RNA transcript of a gene, parts of a gene or a cDNA sequence representing a gene. With reference to a first oligonucleotide, a second oligonucleotide may be referred to as being “upstream” from it; if the second oligonucleotide is positioned nearer to the 5′ end of the first oligonucleotide or “downstream” if the second nucleotide is nearer to the 3′ end of the first oligonucleotide.

Concatemer—It is composed by at least two nucleotide monomers sequences linked end to end, optionally separated by a linker or spacer. For the purpose of the present invention, a concatemer comprises at least two tags, two ditags, two nucleotide fragments or two oligonucleotides prepared according to the method of the invention. In the present invention, two oligonucleotides may be concatenated such that the 5′ to 3′ orientation of one nucleotide fragment in an oligonucleotide is opposite to the orientation of an adjacent nucleotide fragment positioned upstream or downstream of it.

Plasmid—With the term vector or recombinant vector it is intended a plasmid, virus or other vehicle known in the art that has been manipulated by insertion or incorporation of the ditag genetic sequences. Such vectors contain a promoter sequence that facilitates the efficient transcription of the inserted sequence. The vector typically contains an origin of replication, a promoter, as well as specific genes which allow phenotypic selection of the transformed cells. Vectors suitable for use in the present invention include for example, pBlueScript (Stratagene, La Jolla, Calif.); pBC, pZErO-1 (Invitrogen, Carlsbad, Calif.) and pGEM3z (Promega, Madison, Wis.) or modified vectors thereof, as well as other similar vectors known to those of skill in the art. The pGEM vectors have also been disclosed in U.S. Pat. No. 4,766,072, herein incorporated by reference. In the present invention, the plasmid PGIS4a2 (clone B4-1) (FIG. 5A) was used.

Obtain, derive, prepare—to use molecular biology and genetic engineering and manipulation techniques on biological material such as nucleic acids and proteins to confer upon the material certain desired characteristics. The terms obtain, derive and prepare may be used interchangeably under the present invention.

Amplification—increasing the copy number of nucleic acids. One method commonly used is that of polymerase chain reaction (PCR). Other amplification methods known to a skilled person such as bacterial amplification or rolling circle amplification may also be used.

Tag—A tag or signature is an identifiable sequence of nucleic acids. It may refer to either the 5′- or 3′-most terminal nucleic acid sequence (terminus; of any length but usually 18-20 bp) derived from any contiguous DNA region. The terms tag and signature may be used interchangeably under the present invention. Under the present invention, a single tag signature (about 20 bp) from each of two nucleotide fragments may be ligated to form a “tag1-linker-tag2” (also referred to as “first tag-linker-second tag) paired end ditag (PET) structure. Another possible arrangement is a linker-tag-tag-linker structure where a linker flanks a tag (that is, a linker is positioned upstream and/or downstream to at least one of the tag).

Linker—A linker is an artificial sequence of nucleic, usually containing one or more restriction enzyme recognition sites.

Ditag—A short (usually 12-60 bp) strand of nucleotides comprising at least one tag or signature derived from a longer strand of nucleotides. A ditag may be prepared according to US 20050255501 and/or US 20050059022, the contents of which are herein incorporated by reference. A ditag may comprise either or both the 5′ end region (also indicated as 5′ tag) and 3′ end region (also indicated as 3′ tag) of a nucleic acid molecule. Under the present invention, a single tag signature (about 20 bp) from each of two nucleotide fragments may be ligated to form a “tag1-linker-tag2” (also referred to as “first tag-linker-second tag) paired end ditag (PET) structure. When two paired end ditags (PET) each comprises a 5′ tag and a 3′ tag of each nucleotide fragment, for example the structure of “PET1-linker-PET2”, is called a diPET.

Sequencing—The methods used to determine the order of constituents in a biopolymer, in this case, a nucleic acid. Sequencing techniques used include Sanger method and modified variations thereof, as well as pyrosequencing or the “454 method” of sequencing.

In the following description, details are provided to describe the embodiments of the present invention. It shall be apparent to one skilled in the art, however the invention may be practiced without such details. Some of the details may not be described at length so as not to obscure the invention.

For the performance of the methods of the present invention for a particular embodiment, any description disclosed for the purpose of carrying out other embodiments of this invention may also be used and are herein incorporated by reference. In particular, technique(s), reagents, experimental conditions, restriction sites, enzymes, vectors, primers, and the like. In particular, it will be evident to any skilled person how to adapt techniques and material disclosed for the other embodiments to the present embodiment of the invention.

Bibliographic references mentioned in the present specification are for convenience listed in the form of a list of references and added at the end of the examples.

Standard molecular biology techniques known in the art and not specifically described were generally followed as described in standard molecular biology reference books such as Molecular Cloning: A Laboratory Manual by Sambrook and Russell, Third Edition, 2001, published by Cold Spring Harbor Laboratory Press.

### Description

The present invention relates to a new method of manipulating nucleic acids. More specifically, the present invention relates to manipulation of nucleic acids by concatenating them. In particular, the invention relates to methods for the preparation of ditags and/or tags representing polynucleotides by concatenation.

In one aspect, the present invention provides a method for length-controlled concatenation of signature tags representing polynucleotides such that concatemers having desired number of ditags and/or tags or having a particular length may be prepared. The present invention also provides molecules and components prepared by the method.

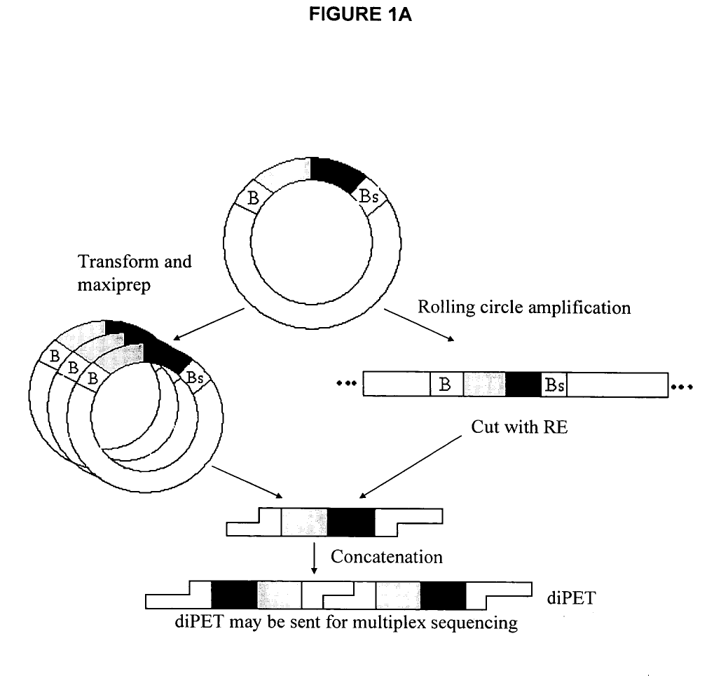

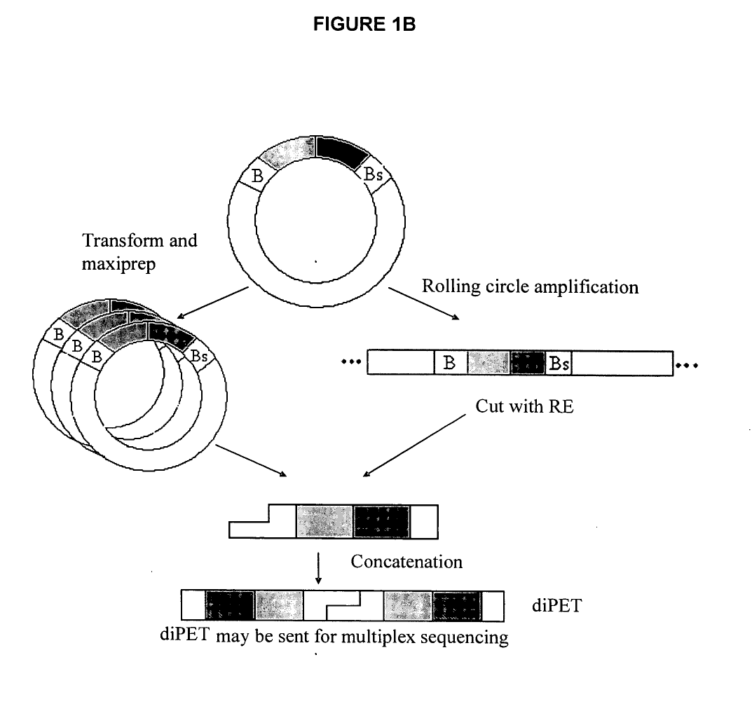

Accordingly, there is provided a method of length-controlled concatenating nucleotide fragments, the method comprising: (a) providing at least two nucleotide fragments, wherein each fragment has one ligatable end and one non-ligatable end; and (b) allowing the two fragments to ligate at the ligatable ends to form at least one oligonucleotide comprising at least two concatenated nucleotide fragments (FIG. 1). This method may be repeated one or more times.

The method above may further comprise treating the at least one obtained oligonucleotide to produce at least one oligonucleotide with two ligatable ends and allowing the oligonucleotide to self-circularize at the ligatable ends. The method may further comprise selecting the at least one circularized oligonucleotide and/or amplifying the oligonucleotide. The method may further comprise treating the circularized and/or amplified oligonucleotide to produce at least one oligonucleotide having one ligatable end and one non-ligatable end, and allowing at least two oligonucleotides to ligate at the ligatable ends to form a concatemer comprising at least two oligonucleotides. This method may be repeated one or more times. In one aspect, the repetition of concatenating results in a doubling of the number of concatenated nucleotide fragments.

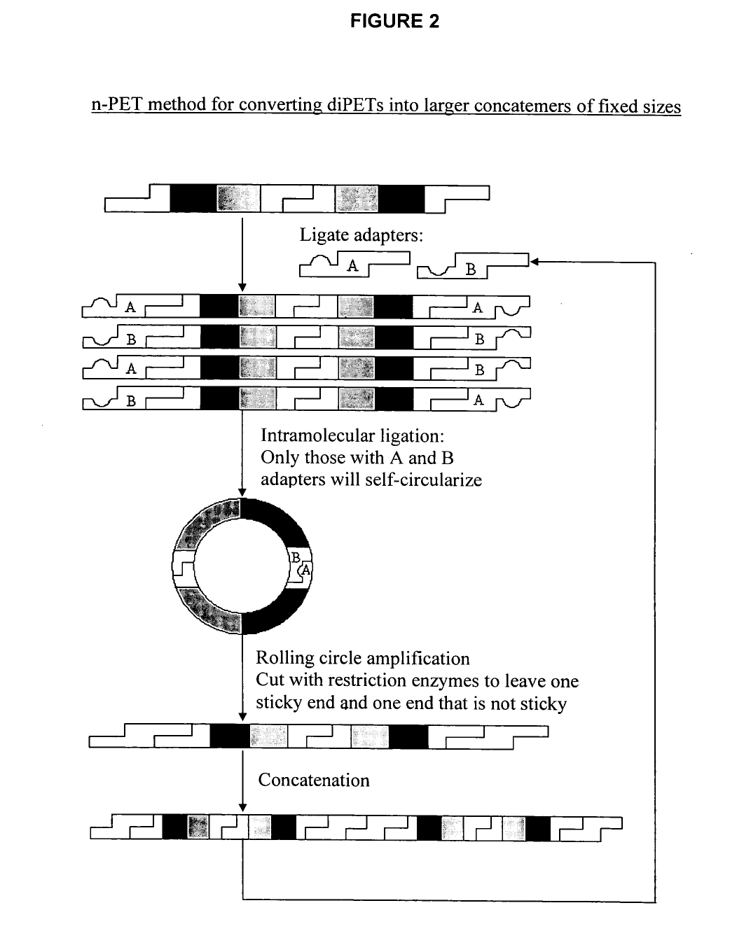

In another embodiment, there is provided a method (see FIG. 2) comprising the steps of (a) providing at least one oligonucleotide comprising at least one nucleotide fragments, preferably comprising at least two nucleotide fragments, wherein the oligonucleotide has ligatable ends; (b) allowing the at least one oligonucleotide to self-circularize at its ligatable ends; (c) selecting the at least one self-circularized oligonucleotide; (d) treating the selected circularized oligonucleotide with at least one restriction enzyme to obtain at least one oligonucleotide with one ligatable end and one non-ligatable end; and (e) concatenating at least two oligonucleotides at the ligatable ends to form a concatemer of at least two oligonucleotides or at least two nucleotide fragments. In particular, the provided oligonucleotide in step (a) may comprise at least two concatenated nucleotide fragments, and the obtained concatenated oligonucleotide in step (e) comprises at least two four-concatenated nucleotide fragments (as shown in FIG. 2). Each nucleotide fragment may comprise at least one ditag, the ditag comprising at least one first tag comprising a 5′ terminus and at least one second tag comprising a 3′ terminus of a polynucleotide. In one embodiment, the first ditag of nucleotide fragment has opposite orientation to the second ditag of the same nucleotide fragment. Accordingly, each nucleotide fragment of the oligonucleotide has the opposite orientation to a nucleotide fragment positioned upstream and/or downstream. This method may be repeated one or more times. In one aspect, each repeating of concatenating results in a doubling of the number of concatenated nucleotide fragments.

### Multiplex Sequencing

The “454” sequencing or pyrosequencing technology has been developed for genomic DNA sequencing but each read can only read 100 bp. This restriction places a limit on the number of tags that can be used. When increasing the number of tags that can be accommodated, the length of each tag will necessarily have to be shortened; concomitantly decreasing specificity and increasing ambiguity. In contrast; if ditags are used in the method of the present invention to prepare length-controlled concatemers, each read will reveal hundreds or even thousands of base pairs of information as demarcated by the ditags. Furthermore, using diPETs as templates, the sequencing throughput and capacity of “454” sequencing are increased two-fold. Hence, the method of the present invention, when applied to preparing concatemers for sequencing and coupled with multiplex sequencing methods, is at least 500-fold more efficient than any of the currently existing methods for DNA sequencing analysis. This method of length-controlled concatenation can be extended for a number of cycles to prepare concatemers having multiple ditags and may be applied to generate desired lengths of concatemers of any kind of tag fragment such as SAGE or “454” multiplex sequencing technology. In addition, the method of the present invention may be applied to cDNA sequencing and ChIP DNA fragment sequencing as well as other sequencing technologies such as SAGE or MPSS.

To overcome the problems and limitations of the current art, the present invention provides, in one embodiment, a method for length-controlled concatenation of signature tags representing polynucleotides such that concatemers having desired number of tags or ditags and having a particular length may be prepared. Such concatemers may be used in various sequencing technologies.

### Tags and Ditags

The ditags of the present invention may be prepared or obtained from the paired end ditagging (PET) strategy (US 20050255501). For the purpose of the present application, a tag is a fragment obtained from a nucleic acid molecule and represents the polynucleotide from which the tag was obtained or derived from. The polynucleotide which is intended to shrink or represent may be RNA, mRNA, genomic DNA, full-length cDNA, or cDNA.

Under the present invention, two tags or fragments that are present in an oligonucleotide of the present invention may also be called a ditag. Like fragments or tags, a ditag is shorter than the original nucleic acid molecule from which it originates or which it represents. Preferably, the ditag must be much shorter than the original nucleic acid molecule. As consequence of the “shrinking”, the ditag may essentially comprise either or both the 5′ end region (also indicated as 5′ tag) and 3′ end region (also indicated as 3′ tag) of the original nucleic acid molecule. Hence, the portion of the original nucleic acid molecule that is between or inside the 5′ tag and 3′ tag is not included in the ditag. The ditag according to the invention retains the most informative features of the original nucleic acid molecule, namely: the start and the end signatures of the nucleic acid.

The 5′ tag and 3′ tag forming the ditag may have the same or different size. Preferably, they have the same number of nucleotides. The ditag may be of any size, but needs to be meaningful and advantageous over the size of the parental sequence from which it is derived. The preferred size of a tag or ditag is determined by genome complexity. For a bacterial genome a tag from about 8 bp to about 16 bp may be sufficient whereas for a complex genome like the human genome, a 16-20 bp tag (which results in a 32-40 bp ditag) may be considered. In general, the size of the ditag is from about 12-60 bp.

For the purpose of the present application, the terms 5′-terminus, 5′-end and 5′-tag are equivalent to each other and may be used interchangeably. In the same way, the terms 3′-terminus, 3′-end and 3′-tag are equivalent to each other and may be used interchangeably. In an original nucleic acid molecule or polynucleotide, or portion inside a nucleic acid molecule or polynucleotide that one intends to reduce or represent, each 5′-end and 3′-end represents a region or portion closest to the extremity; farthest from the middle region of the nucleic acid molecule or polynucleotide. With a 5′ or 3′ terminus of a polynucleotide, it is understood that any region, fragment or whole piece of a polynucleotide that comprises the actual 5′ or 3′ terminus of the polynucleotide are included.

Each ditag comprises sufficient information to characterize a specific polynucleotide. Hence, the ditag is representative of the structure and identity of the polynucleotide.

### Concatenating Oligonucleotides, Nucleotide Fragments, Ditags or Tags

While fragments or tags of sufficient length may be prepared, obtaining concatemers of a predetermined length involve technical difficulties. Current methods for tag concatenation randomly generate concatemers with a range of lengths: monomers, dimers, trimers and so forth. In the current art, such concatemers have to be run in an electrophoresis gel to separate the concatemers, and the desired size of concatemers are excised from the gel—a laborious task. This technique is inefficient and requires large amounts of input DNA.

The present invention provides a method of length-controlled concatenation to generate oligonucleotides of a predetermined length. The present invention achieves this by preparing fragments; ditags or tags with a ligatable end and a non-ligatable end. Using this technique, a compatible, cohesive or sticky end on one fragment or tag will join or ligate to another sticky end on another fragment or tag. When this happens, the non-sticky ends will not permit further ligation and concatenation stops. This technique is further illustrated in the examples below. Should ligatable ends not be found readily in the fragments or tags, suitable adaptors possessing the appropriate restriction enzyme recognition sites may be ligated to the fragments or tags. The ligatable ends may be palindromic ends.

Alternatively, if two different enzymes cannot be used in order to yield one end that is palindromic and another end that will not self-ligate, the enzymes may be used sequentially, and after the first restriction digest, one end may be “blocked” by dephosphorylation or other means, such as attachment to a solid substrate.

### The diPET Embodiment

In this embodiment, tags with the single PET structures in the plasmids are flanked by the restriction enzyme recognition sites for a cohesive palindromic enzyme and an enzyme leaving a blunt end. For example, the two sites may be BamH1 (B) at one side and BseR1 (Bs) at the other side (FIG. 1A), such that the BamH1 cut leaves a palindromic cohesive end compatible to each other, while the BseR1 cut is designed to leave an AA residual or any non-palindrome sequence, which does not match to itself. The PETs may be amplified, whether by bacterial amplification, rolling circle amplification, or other amplification methods (FIGS. 1A and 1B). The PETs are then first cut with one restriction enzyme, in this embodiment BseR1, followed by cutting with a different restriction enzyme, in this embodiment, BamH1.

Released PETs may be purified by any suitable method such as gel purification. Upon exposure to another similarly-prepared PET, any two of the BamH1 cohesive ends will find each other and mate, resulting in oligonucleotide concatemers having a dimer PET or diPET structures with two non-palindromic ends on each side of the oligonucleotide (FIG. 1B). These non-palindromic ends prevent further ligation with other PETs, stopping concatenation.

This embodiment gives rise to a diPET oligonucleotide concatemer made of two PETs of about 80 bp: which is below the maximum capacity of the current “454” sequencing system.

While the preferred embodiment uses PETs, any tag can also be turned into “diPETs” by this method. It is also preferable to use at least one type IIs restriction enzyme, such as BseR1, as this will minimize the length of the border sequences. As long as the cut sites of the type IIs restriction enzyme are different, just one type IIs restriction enzyme site may be used.

### The n-PET Embodiment

This method allows the creation of DNA sequences consisting of n numbers of PETs (where n=4, 8, 16, 32 . . . ), allowing scaling up of the number of PETs added in a length-controlled manner to suit the capacity of the particular sequencing technology used.

Referring to FIG. 2, two different types of adaptors (labeled as A and B) are ligated to the ends of the diPET. These adaptors contain the different restriction sites necessary for restriction digest later. The adaptors are ligated, such that only those diPETs with different adaptors ligated will be circularized by self-circularization, and thus selected by an exonuclease treatment. Rolling circle amplification is performed to amplify the DNA. Alternatively, amplification and selection by PCR is also possible as the adaptor sequences are known. The DNA is then cut with the appropriate restriction enzymes to generate a palindromic end and a non-palindromic end, such that ligation may be used to form a 4-PET oligonucleotide. The cycle may then be repeated as desired to generate larger oligonucleotide concatemers comprising n-PETs.

Alternatively, two different types of adaptors may be ligated to the ends of the diPET, following which PCR is performed. Adaptors which are compatible will snap together, preventing PCR from taking place, allowing only adaptors which are different to be amplified. The DNA may then be cut with the appropriate restriction enzymes, and the cycle repeated if desired.

It may be seen, in the diPET and n-PET embodiments, that concatenation in these embodiments results in the 5′ to 3′ orientation of a nucleotide fragment, tag or ditag being opposite to another nucleotide fragment, tag or ditag adjacent (that is, positioned upstream or downstream) to it (FIGS. 1 and 2). Also seen in this embodiment is the doubling of the number of diPETs concatenated with each repeat of the ligation step.

While the embodiments of the method of the present invention above make use of fragments or tags that have ligatable ends, the method of the present invention can also make use of fragments that do not have ligatable ends by adding suitable adaptors to them. Alternatively, If two different enzymes cannot be used which result in one end that is palindromic and another end that will not match to itself, the enzymes may be used sequentially, and after the first restriction digest, one end may be “blocked” by dephosphorylation or other means, such as attachment to a solid substrate.

The embodiments of the method for fragments, ditags or tags may also be applied to oligonucleotides or oligonucleotide concatemers. For example, suitable adaptors may also be ligated to oligonucleotide concatemers to allow them to ligate to another nucleotide fragment, tag or oligonucleotide with a compatible cohesive end.

Accordingly, in another embodiment, the method may further comprise the steps of treating the at least one oligonucleotide to produce at least one oligonucleotide having one ligatable end and one non-ligatable end, and allowing the oligonucleotide to ligate with a further oligonucleotide or nucleotide fragment to form a concatemer comprising at least two oligonucleotides or at least one oligonucleotide and at least one nucleotide fragment.

In another embodiment, the method may further comprise: (a) treating the at least one oligonucleotide to produce at least one oligonucleotide having two ligatable ends compatible with each other, and allowing the oligonucleotide to self-circularize; (b) selecting the at least one self-circularized oligonucleotide; (c) optionally amplifying the selected oligonucleotide; (d) treating the oligonucleotide from the previous step to produce at least one oligonucleotide with one ligatable end and one non-ligatable end; and (e) allowing two oligonucleotides to ligate at the ligatable ends to form a concatemer comprising at least two oligonucleotides.

There is also provided an isolated oligonucleotide comprising at least two nucleotide fragments, wherein each fragment has at least one ligatable end and and one non-ligatable end, and the fragments are ligated at the ligatable ends to form the oligonucleotide. Preferably, the ligatable ends are palindromic cohesive ends. The concatemer or concatenated oligonucleotide(s) according to the invention comprises at least one nucleotide fragment, the fragment comprising at least one ditag, the ditag comprising at least one first tag comprising a 5′ terminus and at least one second tag comprising a 3′ terminus of a polynucleotide. The polynucleotide may be a full-length cDNA or one or more exons. Accordingly, the ditag may be representative of the full-length cDNA. In one embodiment; the concatemer or oligonucleotide according to the invention has each nucleotide fragment (or ditag) in an orientation opposite to the orientation of a nucleotide fragment (or ditag) positioned upstream and/or downstream. The concatemer, oligonucleotide, nucleotide fragment, ditag or tag according to the invention may be inserted into a plasmid or vector. Accordingly, there is also provided a plasmid or vector comprising at least one concatemer, oligonucleotide, nucleotide fragment, ditag or tag according to the invention. The plasmid or vector may be inserted in a host cell.

There is also provided a kit for concatenating oligonucleotides, nucleotide fragments, ditags and/or tag according to any embodiment of the invention, comprising at least one of a restriction enzyme, at least one nucleotide fragment, ditag or tag, optionally a vector, and any reagents as herein disclosed (for instance as described in the examples) for the reaction of concatenation. The kit may further comprise illustration and/or information pertaining to the use of the kit.

There is also provided a library comprising at least a concatemer, concatenated oligonucleotides, concatenated nucleotide fragments, concatenated ditags and/or concatenated tags according to any embodiment of the invention.

### Variations

For the embodiments in this aspect of the invention, many variations in the method are possible. For example; the tags or nucleotide fragments, and/or oligonucleotides or concatemers may be amplified. The method amplification may be by bacterial amplification, by rolling circle amplification, and/or by polymerase chain reaction. The method may comprise repeating the steps one or more times to obtain concatemers of desired lengths or number of oligonucleotides. The repeating may result in a doubling of the number of oligonucleotides in the concatemers. The ligatable end of each fragment, tag or ditag may be a palindromic cohesive end. The ligatable end and/or the non-ligatable end may be located in at least one adaptor. The adaptor may be part of a plasmid or vector. The nucleotide fragment may comprise at least one ditag, the ditag comprising at least one first tag comprising a 5′ terminus and at least one second tag comprising a 3′ terminus of a polynucleotide. Each nucleotide fragment or tag of the concatemer may have an orientation opposite to the orientation of a nucleotide fragment positioned upstream and/or downstream. The method may further comprise sequencing the concatemer. The sequencing may be by any suitable method, for example, by pyrosequencing.

Having now generally described the invention, the same will be more readily understood through reference to the following examples that are provided by way of illustration and are not intended to be limiting of the present invention.

### EXAMPLES

### Example 1

### BseRI Linearization of Single PET Plasmid

Start from the Maxiprep amplication of the vector performed after transforming single PET plasmid DNA (described in US 20050255501 and US 20050059022). Any suitable vector may be used to clone and amplify the sequence of interest, for example, pGIS4a2 (FIG. 5A; SEQ ID NO:14) or pGIS3h (FIG. 5B; SEQ ID NO:15) Usually, the yield is about 1.2 mg from 10 Q-trays, with a concentration of about 400 ng/ul

| | | |
|---|---|---|
| 1000 ug single PET plasmid DNA | 2500 | ul |
| 10x NEBuffer 2 (New England Biolabs) | 400 | ul |
| 100x BSA (New England Biolabs) | 40 | ul |
| 4 U/ul BseRI (New England Biolabs) | 1000 | ul (4 fold excess) |
| Nuclease-free water | to 4000 | ul |

### For more efficient enzyme digestion, it is advisable to aliquot the reaction mix in tubes of 100 ul each.

BseR1 digest of single PET plasmid maxiprep—Incubate digestion at 37° C. for 3 hours maximum. Perform phenol-chloroform extraction at pH 7.9 using Eppendorf 50 ml phase-lock gel.

Take note of the minimum and maximum volumes required for the various sizes of Phase-Lock gels available. To adjust the volume to the minimum volume required, add up to 1000 μl of nuclease-free water.

50 ml phase-lock gel:

Minimum volume: 5 ml

Maximum volume: 20 ml

Isopropanol precipitation was performed next due to the large volume involved:

| | |
|---|---|
| BseRI digested single PET plasmid | 500 ul |
| 3M NaOAC pH5.2 (Ambion) | 50 ul |
| Glycoblue (Ambion) | 5 ul |
| Isopropanol (Sigma) | 500 ul |
| Total | 1055 ul |

Incubate at −20° C. for an hour. Centrifuge at 13,000 RPM for 30 mins at 4° C. (using a Eppendorf 5415R centrifuge; the same centrifuge may be used through the rest of the protocol) and wash once with 75% ethanol (Prepared from 100% ethanol, from Merck). Resuspend the pellet DNA in a final volume of 1500 ul Qiagen Elution Buffer (EB).

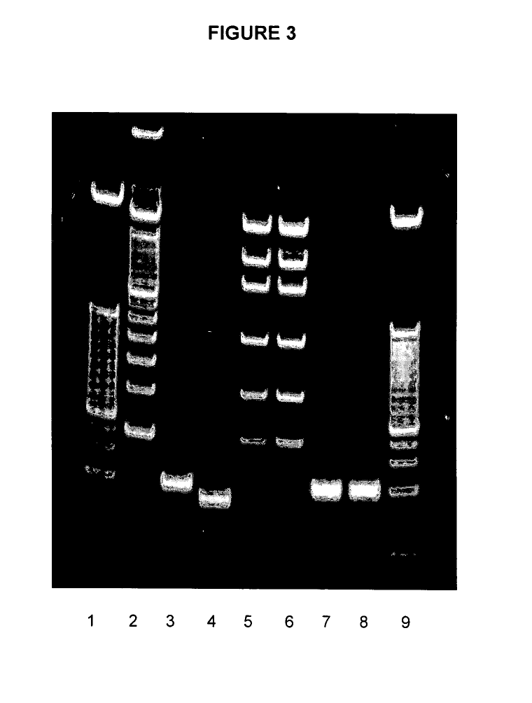

For quality checking, run 400 ng of BseRI cut and uncut single PET plasmid on 1% agarose gel, 110V for 40 mins using the medium gel. The presence of the supercoiled single-PET plasmid would lead to the generation of PETs that are of BamHI cohesive ends on both the 5′ and 3′ ends of the PETs. These would then concatenate and the final product would be a mixture of BamHI PETs concatemers and diPETs of interest. Repeat the BseRI digestion if majority of the supercoiled single PET plasmid remains uncut (FIG. 3).

If it is shown on the gel picture that there digestion is incomplete, i.e. supercoiled single PET plasmid bands are still present, it is advisable to set up another BseRI digestion.

Dephosphorylation of single PET plasmid after BseRI digestion Note: This step is only crucial for diPET generation via the pGIS3h vector. There is no harm in doing this for the pGIS4a vector but it is unnecessary. Save about 400 ng of this sample i.e phosphorylated BseRI linearized single-PET plasmid for subsequent electrophoresis to check the quality of dephosphorylation.

| | | |
|---|---|---|
| BseRI cut single PET plasmid | 1500 | ul (~1000 ug) |
| 10x Antarctic Phosphatase buffer | 600 | ul |
| (New England Biolabs) | | |
| 5 U/ul Antarctic Phosphatase | 1000 | ul (5 fold excess) |
| (New England Biolabs) | | |
| Nuclease-free water | 2900 | ul |
| Total | 6000 | ul |

Perform Isopropanol Precipitation:

| | |
|---|---|
| Dephosphorylated BseRI linearized | 500 ul |
| single-PET plasmid | |
| 3M NaOAc pH 5.2 (Ambion) | 50 ul |
| Glycoblue (Ambion) | 5 ul |
| Isopropanol (Sigma) | 500 ul |
| Total | 1055 ul |

Incubate at −20° C. for an hour. Centrifuge at 13,000 RPM for 30 mins at 4° C. Wash 1× with 75% ethanol. Resuspend the pellet in a final volume of 500 ul EB (Qiagen).

### Take about 400 ng and run 1% agarose gel together with the previously saved, phosphorylated BseRI linearized single PET plasmid.

BamHI Digestion to Release Single PETs

| | | |
|---|---|---|
| Phosphorylated/Dephosphorylated BseRI cut | 500 | ul (~1000 ug) |
| single PET plasmid | | |
| 10x Unique BamHI buffer | 100 | ul |
| (New England Biolabs) | | |
| 100x BSA (New England Biolabs) | 10 | ul |
| 20 U/ul BamHI (New England Biolabs) | 100 | ul |
| Nuclease free water | 290 | ul |
| Total | 1000 | ul |

Aliquot 100 ul per reaction tube. Incubate at 37° C. overnight. Perform ethanol precipitation using glycoblue to reduce the volume to allow for the ease of gel loading.

Prepare 5 Tubes of the Following:

| | |
|---|---|
| BamH1 digested plasmid | 200 ul |
| 100% ethanol (Merck) | 600 ul |
| 3M NaOAc pH 5.2 (Ambion) | 20 ul |
| 1M MgCl₂ (0.0225x of the sample volume) | 4.5 ul |
| (Ambion) | |
| Glycoblue (Ambion) | 2 ul. |

Incubate at −80° C. for at least 30 minutes, followed by a 30 min centrifugation at 4° C. Wash with 75% ethanol once. Resuspend the pellet in a final volume of 350 ul EB. For gel loading, add 100 ul of loading dye and load 45 ul/well and not more than 100 ug of DNA/well on a 2% agarose gel. Electrophorese at 80V for 1.5 hours.

Electroelution

Add 800 ul of sterile milli-Q water to Fermentas Eluta Tubes to hydrate the membrane. These will be used subsequently for electroelution.

Excise the ˜40 bp PETs from the gel, visualizing at 365 nm UV. Discard the 800 ul sterile milli-Q water and place the cut gel slice into the Eluta Tube. Use only one gel slice per Eluta Tube. Fill the tube with 1×TAE buffer without Ethidium Bromide and electroelute at 90V for 30 mins and subsequent reversed polarity for 1 min in the cold room. Ensure that the Eluta Tube is free of air bubbles after adding the buffer.

Collect the eluted PETs in multiple 1.5 ml Eppendorf tubes and centrifuge at 13,000 RPM for 10 mins at 4° C. This is to pellet pieces of agarose that might be present. Pipette whatever is possible and carry out isopropanol precipitation:

| | | |
|---|---|---|
| Eluted DNA | 500 | ul |
| 3M NaOAc (Ambion) | 50 | ul |
| 1M MgCl2 | 11.25 | ul |
| (0.0225x of the sample volume, Ambion) | | |
| Glycoblue (Ambion) | 5 | ul |
| Isopropanol (Sigma) | 500 | ul |
| Total | 1066.25 | ul |

Note:
The 1M MgCl₂ is to aid in the participation of small fragments of DNA. Incubate at −20° C. for an hour. Centrifuge at 13,000 RPM for 30 mins at 4° C. Wash 1x with 75% ethanol. Resuspend the PETs in a final volume of 12 ul EB.

Note: The 1 M MgCl₂ is to aid in the precipitation of small fragments of DNA. Incubate at −20° C. for an hour. Centrifuge at 13,000 RPM for 30 mins at 4° C. Wash 1× with 75% ethanol. Resuspend the PETs in a final volume of 12 ul EB.

4. Quantification of PET DNA

PET DNA can be quantified on a mini 4-20% PAGE gel (Invitrogen). Run 0.2 ul of PET DNA together with 25 bp ladder and Low Mass DNA ladder (both from Invitrogen). The latter will help in the estimation of the PET DNA amount. Currently, 2 ul and 4 ul of Low Mass ladder are used in the quantification. Below shows the preparation for the ladders for per loading:

25 bp Ladder Preparation

1 ul of 1 ug/ul 25 bp ladder+9 ul of EB+2 ul loading dye.

Total 12 ul

Low Mass Ladder Preparation

2 ul Low Mass Ladder Preparation

2 ul of Low Mass ladder+8 ul of EB+2 ul loading dye.

Total 12 ul

4 ul Low Mass Ladder Preparation

4 ul of Low Mass ladder+6 ul of EB+2 ul loading dye.

Total 12 ul

Run the PET DNA to be quantified together with these ladders on a 4-20% mini PAGE gel for 30 mins at 200V and TBE buffer (Ambion).

Stain the PAGE gel with Sybr Green I (Molecular Probes) in TBE buffer (final concentration should be 1×, Ambion) for 10-15 mins.

Note: If there is no difference in the intensity of the DNA bands for quantification, it is advisable to load less of the DNA. A rough estimation would be sufficient as the final DiPET would be run on Agilent Bioanalyzer 500/1000 kit for quantification and sizing for ‘454’ pyrosequencing.

DiPETting Reaction

| | | |
|---|---|---|
| PET DNA (at least 5 ug) | 7 | ul |
| 10x Spermidine buffer | 1 | ul |
| (prepared in-house; below) | | |
| 5 U/ul T4 DNA ligase (Invitrogen) | 1 | ul |
| Nuclease-free water | to 10 | ul |

10× ligation buffer with Spermidine is made up of:

60 mM Tris-HCl pH7.5 (Ambion)

60 mM MgCl2 (Ambion)

50 mM NaCl (Ambion)

1 mg/ml BSA (New England Biolabs)

70 mM Beta-mercaptoethanol (Sigma)

1 mM ATP (Invitrogen)

20 mM DTT (Invitrogen)

10 mM spermidine (Sigma)

Incubate at 16° C. overnight. Adjust volume to 200 ul with nuclease-free water and perform phenol chloroform pH 7.9 extraction.

Perform Ethanol Precipitation as Follows:

| | |
|---|---|
| DiPETs | 200 ul |
| 3M NaOAC (Ambion) | 20 ul |
| 1M MgCl2 (Ambion) | 4.5 ul |
| Glycoblue (Ambion) | 2.2 ul |
| Abs EtOH (Merck) | 800 ul |

Incubate at −20° C. for an hour. Centrifuge at 13,000 RPM for 30 mins at 4° C. Wash 1× with 75% ethanol. Resuspend diPETs in 20 ul EB.

After the diPETting reaction, run 1 ul of the DiPET DNA on Agilent Bioanalyzer DNA 500/1000 kit according to the manufacturer's protocol to estimate the concentration and quantity of the DiPETs available. Then, proceed to any preferred method of sequencing (such as ‘454’ sequencing).

### Example 2

### The n-PET Embodiment as Example

Ligation of two different adaptors. The adaptors may contain source-identifying tags if desired. The adaptors should preferably have one end that is complementary to each other, and ideally, on the other end, the adaptors have sticky ends complementary to sticky ends of the DNA such that ligation will be easy and the adaptors will not ligate to themselves. It is best if the DNA already contains sticky ends, like diPETs. If the DNA is blunt, however, the DNA may be A-tailed with DNA polymerase, and then T-tailed adaptors may be used.

A-Tailing (Not Required If Dipets are Used)

| | |
|---|---|
| 10 mm dATP | 0.5 ul |
| ExTaq polymerase | 0.5 ul |
| 10x ExTaq buffer | 2.5 ul |
| 2 ug of sample | 20 ul |
| Nuclease-free Water | 1.5 ul |

The total volume is 25 ul.

Incubate using in a PCR machine with the following program: 72° C. for 30 minutes, followed by 4° C. forever.

### Perform phenol chloroform extract and ethanol precipitation with glycoblue.

### Resuspend in 12 ul of Elution Buffer.

Ligation of Adaptors

| | | |
|---|---|---|
| DNA (approx 200–1000 ng) | say 5 | ul |
| 10x ligase buffer (with spermidine) | 1 | ul |
| T4 DNA ligase (5 U/ul) | 1 | ul |
| Nuclease-free Water | to 10 | ul |

Incubate at 16° C. for 1-2 hours. Ethanol precipitate DNA.

Phosphorylation of Adaptors

| | | |
|---|---|---|
| DNA | say 20 | ul |
| 10x T4 PNK buffer | 5 | ul |
| 10 mM ATP solution | 5 | ul |
| T4 Polynucleotide kinase (3 U/ul) | 1 | ul |
| Nuclease-free Water | to 50 | ul |

### Incubate at 37° C. for 30 minutes, then heat-inactivate at 70° C. for 5 minutes

Circularization of Adaptors

| | | |
|---|---|---|
| Approximately 200 ng DNA | say 50 | ul |
| 5x Invitrogen ligation buffer (with PEG) | 20 | ul |
| T4 DNA ligase (5 U/ul) | 1 | ul |
| Nuclease-free Water | to 100 | ul |

### The DNA should be diluted to a concentration of approximately 2 ng/ul in the final ligation solution—the dilute solution will favour intramolecular ligation.

### Incubate at 16° C. for 16 hours.

Exonuclease Treatment

| | | |
|---|---|---|
| Adapter-Ligated DNA | say 2 | ul |
| Lambda Exonuclease | 1 | ul |
| Exonuclease I | 1 | ul |
| 10x Lambda Exonuclease buffer | top up to 5 | ul if volume of ligated |
| | | material is less than 5 ul. |
| Nuclease-free Water | To 50 | ul |

### Incubate at 37° C. for 1 hour.

Perform phenol chloroform extraction and ethanol precipitation with glycoblue. Wash once with 75% ethanol. Resuspend in 12 ul of Elution Buffer. No gel purification is necessary but if desired, if may be performed as follows.

Gel Purification

Gel purify the circularized adaptor-ligated DNA by adding of 0.2 volumes of bromophenol blue gel loading buffer. Load 60 ul per well of a medium sized 2% agarose gel. Electrophorese at 80V for 1.5 hours. Extract circularized mate-pairs with a sharp scalpel, with minimal UV light exposure.

Use Qiagen Gel extraction kit if DNA is larger than 150 bp. Use GeBA-flex Elutatubes if DNA is smaller than 100 bp; perform according to manufacturers' protocols.

Restriction enzyme digest (eg, HindIII, as according to manufacturer's protocols)

Ligation Reaction

| | |
|---|---|
| BamH1/BseR1 cut single PET | 12 ul |
| 10x ligase buffer with spermidine | 1.5 ul |
| T4 DNA ligase (5 U/ul) | 1 ul |
| Nuclease-free Water | 0.5 ul |

The total volume is 15 ul. Incubate at 16° C. for 16 hours.

### Adjust the volume to 200 ul with Nuclease-free Water.

### Perform phenol chloroform pH 7.9 extraction with phase lock gel.

### Ethanol precipitate with glycoblue

Quality Control and Quantitation with a PAGE gel

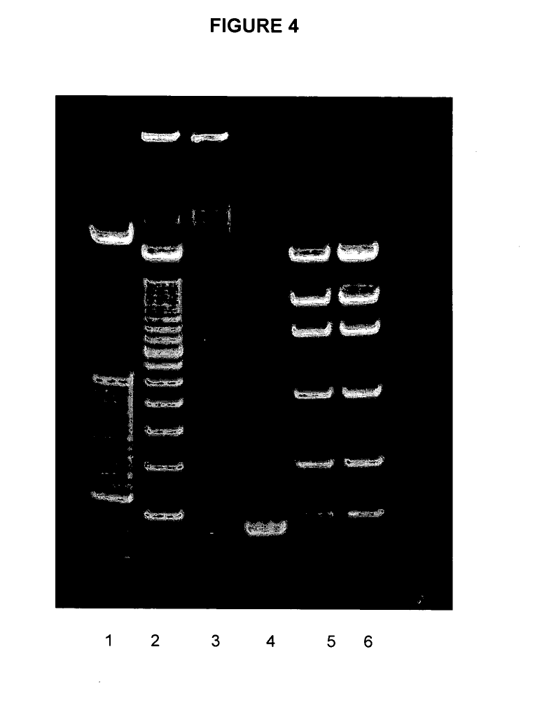

Load approximately 100 ng of sample on a 4-20% gradient PAGE gel, together with Takara Wide Range DNA ladders and Invitrogen Low Mass DNA ladders or other ladders as required for quantitation. Load 1 ul and 2 ul of the Invitrogen Low Mass DNA ladders for more accurate quantitation (FIG. 4).

### Repeat to obtain desired length of concatemer.

PCR modification

If PCR is used for amplification, no exonuclease treatment should be performed. The BD PCR-Select Bacterial Genome Subtraction Kit may be used instead.

### Example 3

### Variation of diPETS or n-PETs Having Cohesive and Non-Palindromic, or Blunt, Ends

This variation is possible by dividing the sample after amplification (Maxiprep or rolling circle amplification or other) into two lots. One lot may be treated with a combination of restriction enzymes, phosphatases and kinases that produce tags with one end that is ligatable, for example a blunt phosphorylated end or a phosphorylated end with an overhang. In this lot, the other end is not ligatable, for example, the other end is a blunt, dephosphorylated end or has either a phosphorylated or dephosphorylated end with an overhang that is not complementary to the first end.

The other lot may be treated with a combination of restriction enzymes, phosphatases and kinases to produce one end that may be ligated, for example a blunt phosphorylated end or a phosphorylated end with an overhang, and another end that cannot be ligated, for example a blunt dephosphorylated end or a phosphorylated or dephosphorylated end with an overhang that is not complementary to the first end, wherein the ligatable end, may be ligated to the ligatable end of the first lot.

Thus, phosphorylated, blunt ends may be ligated to each other, and phsophorylated, cohesive ends complementary to each other may be ligated to each other. For example a digest may produce AA tails in one half, and another digest may produce TT tails in the other half, which can then be mixed in a 1:1 or other suitable ratio to result in dimerization to produce length-controlled diPETs.

### Example 4

### n-PETs with Multiples that are not of 2, 4, 8, 16

First, divide the sample after amplification into any number of aliquots. Then perform the n-pet procedure with these aliquots, repeating any number of times on these aliquots, to end with the production of a blunt, phosphorylated end or a phosphorylated end with an overhang, and another end that cannot be ligated in each aliquot. Ligate suitable aliquots to each other in successive rounds to generate n-PETs with the desired length.

For example, to generate an n-PET consisting of 7 PETs, first separate the sample into 3 aliquots. Then prepare diPETs for two aliquots, and leave one aliquot as it is. With one of the aliquots of diPETs, prepare a 4-PET according to the n-PET method. Finally, combine aliquots: adding the remaining diPET aliquot to the single PET aliquot to form a 3-PET, and then combine the 3-PET with the 4-PET. These steps all require the use of the adapter ligation and selection steps as previously discussed in the n-PET protocol.

### Example 5

### diPETs that have 5′ to 3′ Directionality in Different Orders

First, divide the sample after amplification into two lots. Then treat one lot with a combination of restriction enzymes, phosphatases and kinases such that the 5′ end is a cohesive, non-palindromic end or a blunt end, which may be ligated, while the 3′ end cannot be ligated. Treat the other lot with a combination of restriction enzymes, phosphatases, and kinases such that the 3′ end is a cohesive, non-palindromic end or a blunt end, which may be ligated, while the 5′ end cannot be ligated. Next, combine the two lots and treat with ligase. Fragments from the same lot should not ligate to each other, as their ends are not compatible with each other. However, fragments from different lots should ligate to each other, as their ends have been selected to be cohesive, and thus compatible, with each other.

### Example 6

### Chromatin Interaction Precipitation diPETting (ChIP-PET)

ChIP-PET is a method to identify DNA regions that interact with proteins such as those found in chromatin structures. This variation requires a different vector, pGIS3h (FIG. 5B, SEQ ID NO:15). When cut, this produces diPETs of about 88 base pairs and 4 residues (there are 2 CG residues on either end). There are no AA tails. In contrast, the other variations of the present invention uses pGIS4a2 (FIG. 5A; SEQ ID NO:14). This produces diPETs of a total size of 80 base pairs and 4 residues (there are 2 AA residues on either end).

Although the present invention has been described in detail with reference to examples above, it is understood that various modifications can be made without departing from the spirit of the invention. Accordingly, the invention is limited only the following claims. All cited patents, patent applications and publications referred to in this application are herein incorporated by reference in their entirety.

### REFERENCES

Adams, M., et al., 1991, Science, 252, 1651-1656
Brenner S, et al., 2000, Nature Biotechnology, 18, 630-634
Dunn et al (2002) Genome Research 12(11): 1756-1765
Jongeneel et al., 2003, Proc. Natl. Acad. Sci. USA 100: 4702-4705
Li and Chandrasegaran, Proc. Nat. Acad. Sciences USA 90:2764-8, 1993
Margulies M. et al., Nature, 31 Jul. 2005.
Strausberg, R. L., et al., 1999, Science, 286: 455-457
Velculescu, V. E., et al., 1995, Science, 270, 484-487
U.S. Pat. No. 4,766,072
US 20050255501
US 20050059022

1. A method of length-controlled concatenating nucleotide fragments, the method comprising:
(a) providing at least two nucleotide fragments, wherein each fragment has one ligatable end and one non-ligatable end; and
(b) allowing the two fragments to ligate at the ligatable ends to form at least one oligonucleotide comprising at least two concatenated nucleotide fragments.

2. The method according to claim 1, wherein the ligatable end of each fragment is a palindromic cohesive end.

3. The method according to claim 1, wherein the ligatable end and/or the non-ligatable end is located in at least one adaptor.

4. The method according to claim 3, wherein the adaptor is part of a plasmid or vector.

5. The method according to claim 1, wherein the nucleotide fragments are amplified.

6. The method according to claim 5, wherein the amplification is by bacterial amplification, by rolling circle amplification, and/or by polymerase chain reaction.

7. The method according to claim 1, further comprising the steps of treating the at least one oligonucleotide to produce at least one oligonucleotide having one ligatable end and one non-ligatable end, and allowing the oligonucleotide to ligate with a further oligonucleotide or a nucleotide fragment to form an oligonucleotide comprising more than two concatenated nucleotide fragments.

8. The method according to claim 7, wherein the steps are repeated one or more times.

9. The method according to claim 7, wherein the ligatable end of each oligonucleotide or of the nucleotide fragment is a palindromic cohesive end.

10. The method according to claim 1, further comprising treating the at least one oligonucleotide to produce at least one oligonucleotide with two ligatable ends and allowing the oligonucleotide to self-circularize at the ligatable ends.

11. The method according to claim 10, wherein the ligatable ends are palindromic cohesive ends.

12. The method according to claim 10, wherein the ligatable ends are obtained from two adaptors, each adaptor linked to each end of the oligonucleotide.

13. The method according to claim 10, further comprising selecting the at least one circularized oligonucleotide and/or amplifying the oligonucleotide.

14. The method according to claim 13, further comprising treating the at least one circularized and/or amplified oligonucleotide to produce at least one oligonucleotide having one ligatable end and one non-ligatable end, and allowing the oligonucleotide to ligate with at least a further oligonucleotide at their ligatable ends to form at least two concatenated oligonucleotides.

15. The method according to claim 1, further comprising: (a) treating the at least one oligonucleotide to produce at least one oligonucleotide having two ligatable ends, and allowing the oligonucleotide to self-circularize at its ligatable ends; (b) selecting the at least one self-circularized oligonucleotide; (c) optionally amplifying the selected oligonucleotide; (d) treating the oligonucleotide to produce at least one oligonucleotide with one ligatable end and one non-ligatable end; and (e) allowing the oligonucleotide to ligate with a further oligonucleotide at their ligatable ends to form at least two concatenated oligonucleotides.

16. The method according to claim 15, comprising repetition of the steps (a) to (e) one or more times.

17. The method according to claim 1, wherein the at least one nucleotide fragment comprises at least one ditag, the ditag comprising at least one first tag comprising a 5′ terminus and at least one second tag comprising a 3′ terminus of a polynucleotide.

18. The method according to claim 1, wherein each nucleotide fragment of the concatemer has orientation opposite to the orientation of a nucleotide fragment positioned upstream and/or downstream.

19. The method according to claim 1, further comprising sequencing the concatenated nucleotide fragments.

20. The method according to claim 19, wherein the sequencing is by pyrosequencing and/or by serial analysis of gene expression.

21. A method of length-controlled concatenating nucleotide fragments, the method comprising the steps of:
(a) providing at least one oligonucleotide comprising at least one nucleotide fragment, wherein the oligonucleotide has ligatable ends;
(b) allowing the at least one oligonucleotide to self-circularize at its ligatable ends;
(c) selecting the at least one self-circularized oligonucleotide;
(d) treating the selected circularized oligonucleotide with at least one restriction enzyme to obtain at least one oligonucleotide with one ligatable end and one non-ligatable end; and
(e) concatenating at least two oligonucleotides at the ligatable ends to form a concatenated oligonucleotide comprising at least two nucleotide fragments.

22. The method according to claim 21, wherein the provided oligonucleotide in step (a) comprises at least two concatenated nucleotide fragments, and the obtained concatenated oligonucleotide in step (e) comprises at least four concatenated nucleotide fragments.

23. The method according to claim 21, wherein the nucleotide fragment comprises at least one ditag, the ditag comprising at least one first tag comprising a 5′ terminus and at least one second tag comprising a 3′ terminus of a polynucleotide.

24. The method according to claim 21, wherein the ligatable ends are palindromic cohesive ends.

25. The method according to claim 21, wherein step (e) concatenation is repeated one or more times.

26. The method according to claim 25, wherein each repeat of concatenation results in a doubling of the number of concatenated nucleotide fragments.

27. The method according to claim 21, wherein the ligatable ends are obtained from adaptors.

28. The method according to claim 21, wherein step (c) further comprises amplification of the oligonucleotide.

29. The method according to claim 28, wherein the amplification is by rolling circle amplification, and/or by polymerase chain reaction.

30. The method according to claim 21, the method further comprising sequencing the concatenated oligonucleotide.

31. The method according to claim 21, wherein each nucleotide fragment of the oligonucleotide has orientation opposite to the orientation of a nucleotide fragment positioned upstream and/or downstream.

32. An isolated oligonucleotide comprising at least two nucleotide fragments, wherein each fragment has at least one ligatable end and and one non-ligatable end, and the fragments are ligated at the ligatable ends to form the oligonucleotide.

33. The oligonucleotide according to claim 32, wherein the ligatable ends are palindromic cohesive ends.

34. The oligonucleotide according to claim 32, wherein the fragment comprises at least one ditag, the ditag comprising at least one first tag comprising a 5′ terminus and at least one second tag comprising a 3′ terminus of a polynucleotide.

35. The oligonucleotide according to claim 34, wherein each nucleotide fragment of the oligonucleotide has orientation opposite to the orientation of a nucleotide fragment positioned upstream and/or downstream.

36. The oligonucleotide according to claim 34, wherein the oligonucleotide is inserted into a plasmid or vector.


SEQUENCE LISTING (21 sequences)

SEQ ID NO: 1 - DNA, 33 bp, Artificial
  GsuI-oligo dT primer
gagctagttctggagttttttttttttttttvn

SEQ ID NO: 2 - DNA, 34 bp, Artificial Sequence
  GIS-(N)6 adapter upper strand
ctaaactcgaggcggccgcggatccgacnnnnnn

SEQ ID NO: 3 - DNA, 26 bp, Artificial Sequence
  GIS-(N)6 adapter lower strand
gtcggatccgcggccgcctcgagttt

SEQ ID NO: 4 - DNA, 34 bp, Artificial Sequence
  GIS-(N)5adapter upper strand
ctaaactcgaggcggccgcggatccgacgnnnnn

SEQ ID NO: 5 - DNA, 26 bp, Artificial Sequence
  GIS-(N)5 adapter lower strand
gtcggatccgcggccgcctcgagttt

SEQ ID NO: 6 - DNA, 12 bp, Artificial Sequence
  palindromic upper strand
gtcggatccgac

SEQ ID NO: 7 - DNA, 12 bp, Artificial Sequence
  palindromic lower strand
gtcggatccgac

SEQ ID NO: 8 - DNA, 40 bp, Artificial Sequence
  n-PET TT-tailed adapter (PMR 011) - upper strand
gcttgtaagctactcctcgatgtgctgcaaggcgattaag

SEQ ID NO: 9 - DNA, 42 bp, Artificial Sequence
  N-PET TT-tailed adapter (PMR 11) - lower strand
ttcgaacattcgatgaggagctacacgacgttccgctaattc

SEQ ID NO: 10 - DNA, 41 bp, Artificial Sequence
  n-PET TT-tailed adapater (PMR 012) - upper strand
gcttgtaagctactcctcagcggataacaatttcacacagg

SEQ ID NO: 11 - DNA, 43 bp, Artificial Sequence
  n-PET TT-tailed adapater (PMR 012) - lower strand
ttcgaacattcgatgaggagtcgcctattgttaaagtgtgtcc

SEQ ID NO: 12 - DNA, 22 bp, Artificial Sequence
  PMR011
gatgtgctgcaaggcgattaag

SEQ ID NO: 13 - DNA, 23 bp, Artificial Sequence
  PMR012
agcggataacaatttcacacagg

SEQ ID NO: 14 - DNA, 3531 bp, Artificial Sequence
  pGIS4a2 vector - sense
gggcgaattcgatatcgcggccgcgcctggataaagtcagcagcttccacgccagcttcacacaaaaagtgactgacggtagcggcgcggcggtgcaggaaggtcagggcgatctgtgggtgaaacgtccaaacttattcaactggcatatgacacaacctgatgaaagcattctggtttctgacggtaaaacactgtggttctataacccgttcgttgagcaagctacggcaacctggctgaaagatgccaccggtaatacgccgtttatgctgattgcccgcaaccagtccagcgactggcagcagtacaatatcaaacagaatggcgatgactttgtcctgacgccgaaagccagcaatggcaatctgaagcagttcaccattaacgtgggacgtgatggcacaatccatcagtttagcgcggtggagcaggacgatcagcgcagcagttatcaactgaaatcccagcaaaatggggctgtggatgcagcgaaatttaccttcaccccgccgcaaggcgtcacggtagatgatcaacgtaagtagaggcacctgagtgagcaatctgtcgctcgatttttcggataatacttttcaacctctggccgcgcgtatgcggccagaaaatttagcacagtatatcggccagcaacatttgctggctgcggggaagccgttgccgcgcgctatcgaagccgggcatttacattctatgatcctctgggggccgccgggtaccggcaaaacaactctcgctgaagtgattgcccgctatgcgaacgctgatgtggaacgtatttctgccgtaagtcggatcctcctcgtcgacctgcaggcatgcaagcttgagtattctatagtgtcacctaaatagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttcgataggctccgcccccctgacgagcatcacaaaaatcgacgctcaagtcagaggtggcgaaacccgacaggactataaagataccaggcgtttccccctggaagctccctcgtgcgctctcctgtaccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagaccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttggcattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgtaatacgactcactata

SEQ ID NO: 15 - DNA, 3531 bp, Artificial Sequence
  pGIS4a2 vector - antisense
tatagtgagtcgtattacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatgccaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggtctcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggtacaggagagcgcacgagggagcttccagggggaaacgcctggtatctttatagtcctgtcgggtttcgccacctctgacttgagcgtcgatttttgtgatgctcgtcaggggggcggagcctatcgaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctatttaggtgacactatagaatactcaagcttgcatgcctgcaggtcgacgaggaggatccgacttacggcagaaatacgttccacatcagcgttcgcatagcgggcaatcacttcagcgagagttgttttgccggtacccggcggcccccagaggatcatagaatgtaaatgcccggcttcgatagcgcgcggcaacggcttccccgcagccagcaaatgttgctggccgatatactgtgctaaattttctggccgcatacgcgcggccagaggttgaaaagtattatccgaaaaatcgagcgacagattgctcactcaggtgcctctacttacgttgatcatctaccgtgacgccttgcggcggggtgaaggtaaatttcgctgcatccacagccccattttgctgggatttcagttgataactgctgcgctgatcgtcctgctccaccgcgctaaactgatggattgtgccatcacgtcccacgttaatggtgaactgcttcagattgccattgctggctttcggcgtcaggacaaagtcatcgccattctgtttgatattgtactgctgccagtcgctggactggttgcgggcaatcagcataaacggcgtattaccggtggcatctttcagccaggttgccgtagcttgctcaacgaacgggttatagaaccacagtgttttaccgtcagaaaccagaatgctttcatcaggttgtgtcatatgccagttgaataagtttggacgtttcacccacagatcgccctgaccttcctgcaccgccgcgccgctaccgtcagtcactttttgtgtgaagctggcgtggaagctgctgactttatccaggcgcggccgcgatatcgaattcgccc

SEQ ID NO: 16 - DNA, 2765 bp, Artificial Sequence
  pGIS3H vector - sense
gggcgaattcgatatcgcggccgcgaggattatggatccgactgcagtcggatccatactcctcattgcaggcatgcaagcttgagtattctatagtgtcacctaaatagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttcgataggctccgcccccctgacgagcatcacaaaaatcgacgctcaagtcagaggtggcgaaacccgacaggactataaagataccaggcgtttccccctggaagctccctcgtgcgctctcctgtaccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagaccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttggcattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgtaatacgactcactata

SEQ ID NO: 17 - DNA, 2765 bp, Artificial Sequence
  pGIS3H vector - antisense
tatagtgagtcgtattacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatgccaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggtctcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggtacaggagagcgcacgagggagcttccagggggaaacgcctggtatctttatagtcctgtcgggtttcgccacctctgacttgagcgtcgatttttgtgatgctcgtcaggggggcggagcctatcgaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctatttaggtgacactatagaatactcaagcttgcatgcctgcaatgaggagtatggatccgactgcagtcggatccataatcctcgcggccgcgatatcgaattcgccc

SEQ ID NO: 18 - DNA, 82 bp, Artificial Sequence
  diPET, from PGIS4a2 - sense
nnnnnnnnnnnnnnnnnnnnnnnnnnnnnnnnnngtcggatccgacnnnnnnnnnnnnnnnnnnnnnnnnnnnnnnnnnnaa

SEQ ID NO: 19 - DNA, 82 bp, Artificial Sequence
  diPET, from PGIS4a2 - antisense
aannnnnnnnnnnnnnnnnnnnnnnnnnnnnnnnnncagcctaggctgnnnnnnnnnnnnnnnnnnnnnnnnnnnnnnnnnn

SEQ ID NO: 20 - DNA, 90 bp, Artificial Sequence
  diPET, from pGIS3h - sense
acnnnnnnnnnnnnnnnnnnnnnnnnnnnnnnnnnnnngtcggatccgacnnnnnnnnnnnnnnnnnnnnnnnnnnnnnnnnnnnngtcg

SEQ ID NO: 21 - DNA, 90 bp, Artificial Sequence
  diPET, from pGIS3h - antisense
gctgnnnnnnnnnnnnnnnnnnnnnnnnnnnnnnnnnnnncagcctaggctgnnnnnnnnnnnnnnnnnnnnnnnnnnnnnnnnnnnnca